(19) United States
(12) Patent Application Publication
(10) Pub. No.: US 2006/0088509 A1
(43) Pub. Date: Apr. 27, 2006

(54) Isolated clk-1 -/- cells from clk-1 heterozygous animals and their use in treating oxidative stress disorders

(76) Inventors: Siegfried Hekimi, Montreal (CA); Xing Xing Liu, La Prairie (CA); Ning Jiang, Wappingers Falls, NY (US); Eric Shoubridge, Westmount (CA)

Correspondence Address: CHOATE, HALL & STEWART LLP, TWO INTERNATIONAL PLACE, BOSTON, MA, 02110, US

(21) Appl. No.: 11/245,278

(22) Filed: Oct. 6, 2005

Related U.S. Application Data
(60) Provisional application No. 60/616,350, filed on Oct. 6, 2004.
(60) Provisional application No. 60/679,658, filed on May 11, 2005.

(51) Int. Cl. A61K 35/407; C12N 5/08

(52) U.S. Cl. 424/93.7; 435/325; 435/370

(57) ABSTRACT

The invention relates to the field of oxidative stress disorder and more specifically to isolated cells from clk-1 +/− animals that do not express CLK1. The invention also relates to methods for treating a subject or a diseased tissue in need of treatment for oxidative stress disorder using isolated clk-1 −/− cells from clk-1 +/− animals.

### PRIORITY

This application claims priority to Provisional Patent Application No. 60/616,350 filed Oct. 6, 2004 and to Provisional Patent Application No. 60/679,658 filed May 11, 2005, each of which is incorporated by reference herein in its entirety.

### FIELD OF THE INVENTION

The invention relates to the field of oxidative stress disorder and more specifically to isolated cells from clk-1 +/− animals that do not express CLK1. The invention also relates to methods for treating a subject or a diseased tissue in need of treatment for oxidative stress disorder using isolated clk-1 −/− cells from clk-1 +/− animals.

### BACKGROUND OF THE INVENTION

The power of using the genetic approach to elucidate the mechanisms of aging has been underscored by the possibility of identifying long-lived mutants in invertebrate animal models of aging. Indeed, when a loss-of-function mutation in a gene prolongs lifespan, one has to conclude that the normal function of that gene limits lifespan in the organism under study. In the nematode Caenorhabditis elegans, this approach has been used to identify a number of mechanisms that affect aging: 1) the insulin signaling pathway (Kenyon et al., Nature 366: 461-4, 1993; Kimura et al., Science 277: 942-6, 1997), 2) the clk-1-dependent mechanism (Wong et al., Genetics 139: 1247-59, 1995; Lakowski and Hekimi, Science 272: 1010-3, 1996; Ewbank et al., Science 275: 980-3, 1997), 3) caloric restriction (Lakowski and Hekimi, Proc. Natl. Acad. Sci. USA 95: 13091-6, 1998), 4) a mitochondrial mechanism that alters resistance to oxidative stress and does not affect animal size (Feng et al., Dev. Cell 1: 633-44, 2001; Hekimi and Guarente, Science 299: 1351-4, 2003), 5) a mitochondrial mechanism that acts during development and appears distinct from mechanism 4) in terms of its effects on oxidative stress (Dillin et al., Science 298: 2398-401, 2002; Lee et al., Nat Genet. 25: 25, 2002; Hekimi and Guarente, Science 299: 1351-4, 2003), 6) a pathway linked to germ cell multiplication that might be distinct from the insulin pathway (Hsin and Kenyon, Nature 399: 362-6, 1999), although it involves some of the same molecular players, such as daf-2 and daf-16, 7) a mechanism that has links to telomere length (Benard et al., Development 128: 4045-55, 2001; Joeng et al., Nat Genet 36: 607-11, 2004), and 8) the TOR pathway (Vellai et al., Nature 426: 620, 2003; Jia et al., Development 131: 3897-906, 2004).

In spite of the extensive study of these pathways in invertebrates, in particular C. elegans and Drosophila, and with the exception of caloric restriction, which was discovered in rodents, there is promising but limited evidence as to whether the effects of these pathways on longevity is evolutionarily conserved (Kenyon, Cell 105: 165-8, 2001; Kenyon, Cell 120: 449-60, 2005). In this regard, the best studied pathway is the insulin signaling pathway. One study of mice heterozygous for a knockout of the insulin-like growth factor I receptor (a homologue of DAF-2) found an increase in the lifespan of these animals (Holzenberger et al., Nature 421: 182-7, 2003), and an adipose tissue-specific knockout of the insulin receptor itself is similarly effective (Bluher et al., Science 299: 5724, 2003). On the other hand, although overexpressing catalase in the mitochondria increases mouse lifespan (Schriner et al., Science 308: 1909-11, 2005), another study of mice heterozygous for a knockout that disrupts the function of the manganese superoxide dismutase (sod2), and results in high oxidative stress, failed to reveal an effect on lifespan (Van Remmen et al., Physiol. Genomics 16: 29-37, 2003), in spite of the wealth of evidence supporting the oxidative stress theory of aging.

The gene clk-1, which affects aging and numerous other physiological rates and rhythms in the nematode C. elegans (Wong et al., Genetics 139: 1247-59, 1995), encodes an enzyme that is necessary for the biosynthesis of ubiquinone (co-enzyme Q; UQ) (Marbois and Clarke, J. Biol. Chem. 271: 2995-3004, 1996; Ewbank et al., Science 275: 980-3, 1997; Miyadera et al., J. Biol. Chem. 276: 7713-6, 2001), an essential cofactor in numerous redox reactions, including mitochondrial respiration, as well as a membrane antioxidant, and an oxygen sensor (Georgellis et al., Science 292: 2314-6, 2001). clk-1 mutants accumulate the biosynthetic intermediate demethoxyubiquinone (DMQ) instead of ubiquinone, but also contain ubiquinone of dietary origin, which is necessary for their survival (Jonassen et al., Proc. Natl. Acad. Sci. USA 98: 421-6, 2001; Hihi et al., J. Biol. Chem. 277: 2202-6, 2002). clk-1 mutants have low levels of reactive oxygen species (ROS)(Shibata et al., Science 302: 1779-82, 2003; Kayser et al., Mech. Ageing Dev. 125: 455-64, 2004), and, as a result, low levels of oxidative damage to lipoproteins and decreased activation of oncogenic ras signaling (Shibata et al., Science 302: 1779-82, 2003).

A complete knockout of mclk1, the murine homologue of clk-1, leads to embryonic lethality as well as to a complete absence of ubiquinone in embryos and in mclk1 −/− embryonic stem (ES) cells (Levavasseur et al., J. Biol. Chem. 276: 46160-4, 2001). It also severely affects the activity of mitochondrial complex II, but not complex I and III. The lethality appears to be due to a developmental defect of the placenta. Heterozygous animals, however, are completely viable and newborns have normal levels of ubiquinone, suggesting that mclk1 is fully recessive for ubiquinone biosynthesis.

In view of the lethality of clk-1 −/− knockout animals, there exists a need to develop new tools and methods to impart the benefits of clk-1 −/− cells, e.g., for treating oxidative stress disorders.

### SUMMARY

Therefore, an object of the present invention is to provide tools for treating oxidative stress disorders.

More specifically, that object is achieved by providing an isolated cell from a clk-1 +/− animal, said cell having a clk-1 −/− genotype.

The invention also relates to a composition for use in the treatment of an oxidative stress disorder, comprising an isolated cell of the invention and a pharmaceutically acceptable carrier.

The invention also relates to a method for treating a subject or diseased tissue in need of treatment for an oxidative stress disorder, said method comprising administering a therapeutically effective amount of an isolated clk-1 −/− cell of the invention or a composition of the invention.

The invention further relates to a clk-1 +/− non-human animal comprising clk-1 −/− cells.

### BRIEF DESCRIPTION OF THE FIGURES

FIG. 1: Reduction in the level of DNA damage in mclk1 −/− ES cells and in mclk1 +/− mice. a) DNA damage measured by the comet assay. Staining is for DNA and the presence of a tail associated with a nucleus signals the presence of fast-migrating damaged DNA. Two independent fields of view are shown for each genotype. Many fewer mclk1 −/− ES cells show nuclei with tails, and the tails are smaller. b) The number of nuclei with tails, without consideration of the size of the tails, was determined for mclk1 −/− and mclk1 +/+ES cells (3 samples of 100 cells for each genotype) as well as for liver cells of mclk1 +/− and mclk1 +/+ mice (n=7 mice for each genotype; 3 samples of 100 cells for each mouse). Error bars represent the standard deviation of the means.

FIG. 2: Increased lifespan of mclk1 +/− mice. Kaplan-Meier survival curves are shown with p values calculated by the Mantel-Haenszel logrank test. a) Lifespan extension in the 129SV/j genetic background. mclk1 +/− mice (n=10) lived on average 15% longer than their wild type (n=12) littermates (824.8±103.8 vs 720.2±96.1 days; p=0.00045). All animals were female. b) Lifespan extension in the 129SV/j×Balb/c background. mclk1 +/− mice (n=9) live on average 31% longer than their wild type littermates (n=5) (980.4±105.9 vs 749.8±57.2 days; p=0.00025). c) Lifespan extension in the C57BL/6 background. There are both males and females in the C57BL/6 study and both sexes behave similarly. Although the study in the C57BL/6 background is not finished, the available data shows a median survival of 686 days for mclk1 +/+ (n=5) and of 821.5 days for mclk1 +/− (n=8), a difference that is already significant at p=0.00345. Currently, the median lifespan of the males is 726 days for mclk1 +/+ (n=3) and 837 days for mclk1 +/− (n=5) (p=0.026).

FIG. 3: Groups of cells lacking mCLK1 expression can be observed in the livers of mclk1 +/− mice with extended longevity. Immunohistochemical analyses with anti-mCLK1 antibody revealed that groups of cells lacked mCLK1 expression in the livers of old mclk1 +/− mice only. Uniform staining is seen in young (5 months old) mclk1 +/+ (a) and mclk1 +/− (d) mice. However, while there is uniform staining in 25 months old mclk1 +/+ mice (b), the staining is patchy in similar mclk1 +/− mice (e). Large groups of cells without staining surround the central veins (arrows in e) and appear to expand throughout the whole classical hepatic lobule, which is the region drained by a central vein. Other central veins appear surrounded by mCLK1-positive cells only (e.g. lower arrow in e). RNA in situ Hybridization (RISH) with antisense DIG labelled probe for mclk1 similarly showed uniformly positive cells in 25 months old mclk1 +/+ mice (c), but in similar mclk1 +/− mice (f) there were groups of cells that either lacked (e.g. left arrow) or expressed (e.g. right arrow) the signal for the mclk1 transcripts (blue). The nuclei (pink) were counterstained by nuclear fast red. A minimal decrease in the expression of three mitochondrial protein markers (SOD2, cytochrome C and subunit 1 of complex IV) was found to accompany the loss of mCLK1 expression. g, h, and i show liver sections stained with the mCLK1-specific antibody, and j, k and l show corresponding adjacent sections using antisera against SOD2 (j), cytochrome c (k) and subunit 1 of complex IV (l). The a and b symbols in g and j, and in h and k, identify similar points in adjacent sections.

FIG. 4: Loss-of-heterozygosity (LOH) at the mclk1 locus. Laser-capture microdissections (LCM) of groups of 20-30 cells were obtained from mCLK1-negative (lanes labeled—in the figure) or mCLK1-positive (lanes labeled + in the figure) regions of sections from livers of old mclk1 +/− animals stained for the mCLK1 protein by immunocytochemistry. DNA isolated from these cells was then amplified by whole-genome multiple strand displacement amplification (MDA). Amplified DNA was used for PCR amplification with mclk1-specfic primers. This yields two products from mclk1 heterozygous DNA, one corresponding to the wild-type gene (300 bp) and a larger one corresponding to the disrupted allele (600 bp). a) DNA specifically corresponding to the wild-type mclk1 allele is lost from cells that do not express mCLK1. Lane 1: Negative control provided by LCM buffer, without any captured cells, but which subsequently underwent all procedures (DNA extraction, MDA and PCR). Lane 2: PCR from captured cells expressing mCLK1. Lane 3: PCR from captured cells not expression mCLK1. Lane 4: PCR production from DNA of a wild-type mouse tail (positive control). b) Control for extracted DNA quality. Wild type DNA from the igf1r locus (on chromosome 11) and p53 locus (on chromosome 7) can be unfailingly PCR-amplified from both mCLK1 negative and mCLK1 positive cells. The same sample obtained by LCM and whole genome MDA is being used for PCR in lanes 1, 3, and 5 from a mCLK1-positive group of cells, and in lanes 2, 4, and 6 from a mCLK1-negative group of cells.

FIG. 5: Quinones in mclk1 +/− mice. a) Reverse-phase HPLC chromatograms show the elution of UQ6, DMQ9 and UQ9 standards, and the elution of quinones from representative livers of mclk1 +/− and an mclk1 +/+ mice. UQ6 is added in the liver samples as an internal standard. No DMQ9 peak was detected in any of the liver samples from mclk1 +/− animals (n=7; age range 14-22 months). b) Ubiquinone levels in livers and kidneys of mclk1 +/− mice. In the livers, but not in the kidneys, ubiquinone levels were significantly decreased compared to that in wild-type littermates (n=7 for each genotype; 3 measurements were taken for each liver; P=0.0024). The error bars represent the 95% confidence interval (˜2× the standard error of the mean).

FIG. 6: Sensitivity of mclk1 −/− ES cells to cell death-inducing agents. We tested serum starvation (48 hours) and treatment with etoposide (20 μM, 24 hours), anisomysin (2 μM, 24 hours), staurosporine (0.4 μM; 24 hours), all-trans retinoic acid (1 μM, 96 hours) and sodium azide (15 μM, 24 hours). Cells were seeded in six-well dishes at 1×10⁵/well in ES cell medium with or without compound and analyzed by the trypan blue exclusion method. mclk1 −/− cells were neither resistant nor hyper-sensitive to sodium azide and staurosporine but, in addition to their resistance to menadione, these cells were resistant to etoposide, anisomycin, all-trans retinoic acid, and serum withdrawal. However, upon treatment with sodium pyruvate, which partially rescues growth rate (TABLE 1), the resistance of the mclk1 −/− cells became indistinguishable from that of the mclk1 +/+ cells, suggesting that the resistance of the untreated cells is entirely due to their slow growth rate.

FIG. 7: Normal growth and body weight of mclk1 +/− in the 129SV/j background. The weights of male and female animals were measured monthly. mclk1 +/− and +/+ mice were littermates. The weights and the growth rate of females mclk1 +/− and +/+ mice appear indistinguishable. The sample size for each time point varies from 4 to 15 for females and from 1 to 13 for males. The error bars represent the standard deviations. Due to the limits of the dataset, further data will be needed to confirm the apparent larger weight of old heterozygous males.

### BRIEF DESCRIPTION OF THE INVENTION

The present invention has yielded the unexpected discovery that clk-1 +/− mice have an increased lifespan that is accompanied by a decrease in oxidative damage to DNA. In the livers of old, long-lived, clk-1 +/− mice, large groups of cells do not express CLK1, and frequently fill an entire hepatic lobule, suggesting that they arose clonally through a mechanism of loss-of-heterozygosity followed by positive selection due to their increased stress resistance. Moreover, the applicants have shown that clk-1 −/− mouse embryonic stem cells have low levels of reactive oxygen species, increased resistance to oxidative stress-dependent apoptosis, and reduced oxidative damage and are thus useful for treating oxidative stress disorders.

The results presented herein demonstrate that reducing clk-1 activity reduces reactive oxygen species (ROS) levels, oxidative stress, and oxidative damage in mouse cells, and prolongs the lifespan of whole animals. The fact that reduction of CLK1 activity has similar ROS-reducing and lifespan-lengthening effects in mice and in nematodes, and that the clk-1 gene product is an enzyme involved in the production of a major cellular redox cofactor strongly show a causal link between oxidative stress and aging.

It is therefore an embodiment of the invention to provide an isolated cell from a clk-1 +/− animal and that cell of the invention has a clk-1 −/− genotype. The term “isolated”, when used in reference to a cell means that a naturally occurring clk-1 −/− cell has been removed from its normal tissual (e.g., organ) environment. Thus, the cell may be in a solution or placed in a different environment.

According to a preferred embodiment, the cell of the invention consists of embryonic stem cells. As another preferred embodiment, the present invention contemplates providing clk-1 −/− cells which consist of non-embryonic cells such as, but not limited to, liver cells, skin cells, and intestine cells. For instance, the non-embryonic cells of the invention may consist of stem cells.

Moreover, the isolated cell of the invention has advantageously at least one of the following properties:

low levels of reactive oxygen species,

increased resistance to oxidative stress dependent apoptosis, and

reduced oxidative damage.

As previously mentioned, the isolated cells of the invention are obtained from a clk-1 +/− animal which is preferably in the latter half of its natural life span. Indeed, old clk1 +/− animal are preferred since the presence of large groups of cells that do not express CLK1 were observed in the livers of every old clk1 +/− animal examined.

The current invention can be useful in treating a subject or diseased tissue in need of treatment of an oxidative stress disorder. Therefore, other embodiments of the invention are to provide a composition and a method for treating a subject or diseased tissue in need of treatment for an oxidative stress disorder. The method of the invention comprises the step of administering a therapeutically effective amount of an isolated clk-1 −/− cell or a composition as defined above to the subject or diseased tissue.

As used herein, the term “treating” refers to a process by which the symptoms of a disease associated with an oxidative stress disorder are alleviated or completely eliminated. As used herein, the term “preventing” refers to a process by which symptoms of a disease associated with an oxidative stress disorder are obstructed or delayed.

The composition of the invention for use in the treatment of an oxidative stress disorder comprises an isolated cell of the invention and a therapeutically acceptable carrier. By the phrase “therapeutically acceptable carrier”, it is meant a carrier medium which does not interfere with the effectiveness of the biological activity of the active ingredients of the composition and which is not toxic to the host or patient. Furthermore, the carrier is advantageously a compound with minimum probability of being rejected by the immune system of the subject being treated. Suitable carriers are of common knowledge to one skilled in the art and will not be further detailed.

The amount of isolated clk-1 −/− cells of the invention is preferably a therapeutically effective amount. A therapeutically effective amount of isolated clk-1 −/− cells of the invention is the amount necessary to allow the same to perform their biological role without causing overly negative effects in the host to which the composition is administered. The exact amount of isolated clk-1 −/− cells of the invention to be used and the composition to be administered will vary according to factors such as the type of oxidative stress disorder being treated, the mode of administration, as well as the other ingredients in the composition.

The phrase “oxidative stress disorder,” as used in the current context, arises from an imbalance of cellular pro-oxidant and antioxidant processes resulting in cell death. Oxidative stress has been implicated in a variety of pathological and chronic degenerative processes including the development of cancer, atherosclerosis, inflammation, age-related disorders, neurodegenerative disorders (such as amyotrophic lateral sclerosis (ALS) and Alzheimer's Disease), cataracts, retinal degeneration, drug action and toxicity, reperfusion injury after tissue ischemia, and defence against infection. As also used herein, the term diseased tissue may be used to mean individual cells, as cultured in vitro, or excised tissue in whole or in part. Diseased tissue may also be used to mean tissue in the subject that is undergoing the degenerative process, or tissue within the same organ that may not yet be affected by the degenerative process. The normal tissue may or may not be adjacent to the degenerative tissue.

It will be understood that the treatment envisioned by the current invention can be used for patients with a pre-existing oxidative stress disorder, or for patients pre-disposed to an oxidative stress disorder. Additionally, the method of the current invention can be used to correct cellular or physiological abnormalities involved with an oxidative stress disorder in patients.

According to a preferred embodiment, the isolated cell of the invention is isolated from a cell sample obtained from a donor. For example, the source of the cells can be xenogeneic to the subject to be treated, but preferably the cells are allogeneic, and most preferably the cells are immunologically compatible with the subject to be treated. According to another embodiment of the invention, the isolated cell of the invention is derived from the same type of organ or tissue in need of treatment.

As one skilled in the art may appreciate, the isolated cells of the invention that are immunologically compatible or not with the subject, may be cultured in vitro prior to administer them to the subject. It will be understood that it is well within the general knowledge of the person of the art to choose the suitable which culture method is suitable in accordance with the present invention. Therefore, these methods will not be further detailed.

By the phrase “immunologically compatible,” as used herein, is meant that the cells are obtained from a histocompatible donor in order to minimize the probability of rejection by the immune system of the subject being treated. Preferably, the cells are from an individual who has the same or a compatible HLA phenotype.

If for any reasons, the cells of the invention are not immunologically compatible with the subject, one may alternatively immunologically protect the cells of the invention if such is a suitable process that would still enable to treat the subject against an oxidative stress disorder. By “immunologically protecting” the cells of the invention, it is meant to refer to the encapsulation, containment or other physical separation of a clk-1 −/− cell of the invention from the body into which it is implanted such that the cell is not exposed to and cannot be eliminated by the immune system of the body of the subject, such that cells which are immunologically protected are administered in a manner that physically isolates them from the subject's immune system.

Another embodiment of the invention is to provide a clk-1 +/− non-human animal comprising clk-1 −/− cells. The non-human animal of the invention is preferably characterized by an increased lifespan in comparison to a clk-1 +/+ animal of the same species. The clk-1 +/− animal of the invention, which is for instance, but not limited to, a pig, a cow, a sheep and a mouse, can be advantageously used as a donor for clk-1 −/− cells of the invention or may be used as a tool for research purposes.

The present invention will be more readily understood by referring to the following examples. These examples are illustrative of the wide range of applicability of the present invention and are not intended to limit its scope. Modifications and variations can be made therein without departing from the spirit and scope of the invention. Although any methods and materials similar or equivalent to those described herein can be used in the practice for testing of the present invention, the preferred methods and materials are described.

### EXAMPLES

Inactivation of the Caenorhabditis elegans gene clk-1, which is required for ubiquinone biosynthesis, increases lifespan by an insulin signaling-independent mechanism. The inventors found that homozygous inactivation of mclk1, the mouse orthologue of clk-1, yields ES cells that are protected from oxidative stress and damage to DNA. Moreover, in the livers of old mclk1 +/− mice, hepatocytes that have lost mclk1 expression by loss-of-heterozygosity undergo clonal expansion, suggesting that their resistance to stress allows them to outcompete cells that still express the gene. mclk1 +/− mice, whose growth and fertility are normal, also display a substantial increase in lifespan in each of three different genetic backgrounds. These observations indicate that the distinct mechanism by which clk-1/mclk1 affects lifespan is evolutionarily conserved from nematodes to mammals and is not tied to a particular anatomy or physiology.

Materials and Methods

Cell Culture

Embryonic Stem cells were grown in high glucose Dulbecco's modified Eagle's medium (-pyruvate, -glutamine) supplemented with 20% fetal bovine serum, glutamine, β-mercaptoethanol and Leukemia Inhibitory Factor (LIF) on feeder-free, gelatin coated dishes at 37° C. in an atmosphere of 5% CO₂ and 95% air.

Growth Rate and Cell Death Assay

Cells were seeded in six-well dishes at 1×10⁵/well in ES cell medium with or without supplements (UQ9: 0.16 μM, sodium pyvurate: 110 mg/L). At different time points, the cells were trypsinized and counted with a hemocytometer. The cells were also analyzed by the trypan blue exclusion method 24 hours after treatment with menadione (6 μM).

LIF Requirement Assay

ES cells were plated at a density of 500 cells/cm² in gelatin-coated 6-well plates in ES cell medium containing various concentration of LIF. Three days after inoculation, the proportion of undifferentiated colonies was determined after scoring the morphology of 300 randomly chosen colonies.

Reactive Oxygen Species Measurement

For the fluorimetric measurement of ROS, cells were incubated with 10 μM DCHF-DA (Molecular Probes) for 20 min at 37° C. ROS levels were measured fluorimetrically with excitation and emission wavelenghts of 495 nm and 530 nm, respectively. Oxidative stress was induced by incubating the cells loaded with DCHF-DA with 0.1 mM FeSO₄ and 0.2 mM sodium ascorbate.

Glutathione Measurement

Total cellular glutathione was determined on a deproteinized cell extract as follows: 1.0 ml of buffer (0.1M potassium phosphate and 0.001M EDTA at pH7.0), 100 μl sample, 50 μl NADPH (4 mg/ml), 20 μl DTNB [5,5′-dithiobis (2-nitrobenzoic acid), 0.3 mg/ml], 20 μl glutathione reductase (6 units/ml) were mixed and the linear increase in absorbance at 412 nm was measured.

DNA Damage Assay

DNA damage of cells was measured by using a single-cell gel electrophoresis assay (CometAssay, Trevigen, Gaithersburg, Md., USA) according to the manufacturer's instructions. The Comet tails were scored by examining the fixed and stained cells under a fluorescence microscope with ×10 Planoapo objective. 100 cells were scored per sample.

Lipid Peroxidation Assay

Lipid peroxidation was measured using a TBARS assay kit (ZeptoMetrix, Buffalo, N.Y., USA) according to the manufacturer's instructions. A standard curve was generated by using known amounts of malondialdehyde (MDA).

Mitochondrial Activity

Mitochondrial complex II activity was measured as follows: mitochondria (containing 50 μg protein) were pre-incubated with reaction buffer (25 μM potassium phosphate, 5 μM MgCl₂, 20 μM succinate) for 10 minutes at room temperature. The reaction was started by adding 2 μg/ml Antimycin A, 2 μg/ml Rotenone, 2 mM KCN, 50 μM 2,6-Dichlorophenolindophenol (Sigma) in the presence or absence of 65 μM Q1. The linear decrease in absorbance at 600 nm was recorded (ε=21 mM-1 cm⁻¹).

Animals

The mclk1 knockout mutant was described previously (Levavasseur et al. J. Biol. Chem. 276: 46160-4, 2001) and was maintained in the heterozygous state in the 129SV/j genetic background. By mating mclk1 +/− males to 129SV/j or Balb/c wild-type females, we generated isogenic mclk1 +/− and mclk1 +/+ littermates. mclk1 +/− in the C57B6/L background were obtained by backcrossing mclk1 +/− animals in the 129SV/j background to C57BL/6 animals 6 times, and then maintaining them for by brother/sister matings. All the animals were housed in a pathogen free facility at McGill University and were given a standard rodent diet and water ad libitum. The mice were separated from their mother at 21 days of age and housed 3-5 per cage, with both genotypes present in each cage. Lifespan was determined by recording the age of spontaneous death, or when one of the following criteria was met: unresponsiveness to touch, slow respiration, coldness to touch, a hunched up position with matted fur, or any sign of sudden weight loss.

The onset of fertility was determined by mating mclk1 +/− and mclk1 +/+ female mice from 28 days onwards with fertile wild-type males. The estrus cycle was determined by observing sexual behaviors, recording vaginal plugs as well as the resulting pregnancies and offsprings, and by examining vaginal smear histologically (daily for 2 weeks at the age of 6 months).

Statistical Analysis of Survival

Survival was graphed by the Kaplan-Meier method and analyzed by the Mantel-Haenszel test, which is a logrank test designed to test the difference between two survival curves. The one-tailed p value is presented because the hypothesis being tested is that mclk1 +/− animals live longer, not longer or shorter, than mclk1 +/+ animals. For the completed lifespan studies the mean and standard deviation are presented because the magnitude of the effect of mclk1 +/− on survival could be different at different ages. For the unfinished study (in the C57BL/6 background) only the median survival is presented as the lifespan of the most long-lived animals is unknown.

Immunohistochemistry

Immunohistochemical analysis of mCLK1 expression was performed on formalin-fixed paraffin sections (4 μm) of livers from mice sacrificed at 5 months of age or at natural death. The anti-mCLK1 serum was described previously (Levavasseur et al. J. Biol. Chem. 276: 46160-4 2001) and the anti-Complex IV, anti-Cytochrome C, and anti-SOD2 antibodies were obtained commercially. All antibodies were used at a 1:100 dilution. The avidin-biotin-peroxidase method was used for visualization with 3,3′-diaminobenzidine-tetrahydrochloride as substrate, producing a brown reaction product. For the negative control primary antibody was replaced with non-immunized rabbit serum.

In Situ Hybridization

A mclk1 cDNA was cloned into the RNA expression vector pSPT18 and labeled with DIG following standard procedures. The vector was linearized to allow in vitro run-off synthesis of both sense- and antisense-oriented RNA probes. Paraffin-embedded tissue sections were subjected to a non-radioactive RISH using the DIG-labeled antisense mclk1 probes. Hybridization with the corresponding sense probes served as control. Sections were incubated with Anti-Dioxigenin-AP and NBT/BCIP color developing solution to visualize the mclk1 transcript signal. 0.1% nuclear Fast Red was used for counterstaining.

Laser-Capture Microdissection and DNA Extraction, Whole Genome Amplification and PCR

A PixCell lie Laser Capture Microdissection System (Arcturus, USA) was used to pick up 20-30 cells from mCLK1-negative or mCLK1-positive regions of liver sections from old mclk1 +/− animals stained for the mCLK1 protein by immunocytochemistry. DNA was then isolated with a PicoPure DNA extraction kit (Arcturus, USA).

To use the minute quantities of DNA that are available through LCM, a whole genome multiple strand displacement amplification (MDA) was performed with the GenomiPhi DNA amplification kit (Amersham Bioscience, USA). PCR with amplified DNA was carried out using specific primers for mclk1, igf1r and p53.

HPLC Analysis

Quinones were extracted as described (Miyadera et al., J. Biol. Chem. 276: 7713-6, 2001), with slight modifications. Briefly, the quinones extracted in ethanol and hexane were evaporated with a freezing Speed Vac dryer and kept frozen at −80° C. Shortly after reconstitution with mobile phase (70% methanol and 30% ethanol), the samples were loaded on a reverse phase column (Inertsil ODS-3 C8-3, Ph-3, GL Science) and elution was monitored by a UV light detector at 275 nm. The amount of quinones was determined by comparison to known standards using the 32 Karat software (Beckman Coulter Inc., Fullerton, Calif., USA).

### Example 1

### Phenotypic Analysis of MCLK-1 −/− ES Cells

The inventors have derived mclk1 −/− ES cells by cultivating mclk1 −/− blastocysts derived from mclk1 +/− mothers. A mclk1 +/+ line from the same mothers was also derived and serves as control. In addition to the absence of ubiquinone and the mitochondrial respiration defect observed previously, the inventors have now characterized a number of additional phenotypes of mclk1 −/− ES cells, including: 1) slow cell multiplication, 2) reduced tendency to differentiate in the presence of low levels of leukemia inhibitory factor (LIF), 3) low levels of basal and induced ROS measured by dye-dependent fluorescence 4) resistance to apoptosis induced by the ROS-generating compound menadione (TABLE 1).

To further explore these phenotypes mclk1 −/− and control cells were treated with ubiquinone, or sodium pyruvate, or both. Sodium pyruvate promotes the growth rate of cells with mitochondrial impairment by facilitating the regeneration of cytosolic NAD+ (King and Attardi, Science 246: 500-3, 1989). Treatment with exogenous UQ at 0.16 μM did not reconstitute normal intracellular levels of UQ, which remained at least 4-times lower than in wild-type cells (1.12×10−5 vs 5.73×10−5 nmol/mg protein); yet exogenous UQ was able to reach deep intracellular sites, like the mitochondrial matrix, as indicated by its effect on restoring the function of mitochondrial complex II (TABLE 1). Treatment with either UQ or sodium pyruvate ameliorated the low respiration, slow growth, and LIF resistance phenotypes, and treatment with both compounds almost completely rescued these phenotypes. However, resistance to menadione and ROS levels were unaffected (TABLE 1). This shows that the low ROS levels and concomitant resistance to menadione-induced oxidative stress are not the result of low respiration or slow growth rate.

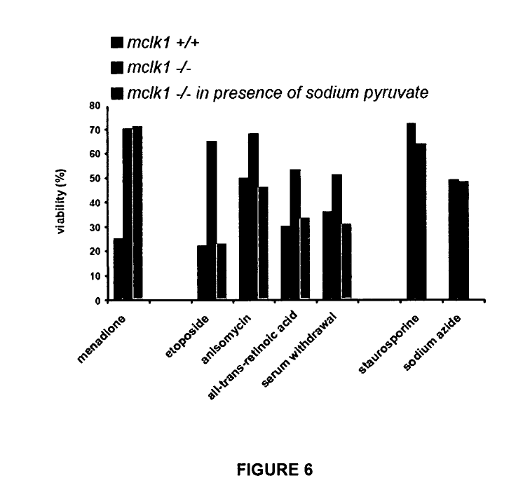

To reinforce the inventors' conclusion that resistance to menadione is not secondary to other phenotypes, the inventors tested the effects of one treatment (serum starvation) as well as of a number of compounds (etoposide, anisomysin, staurosporine, all-trans retinoic acid and sodium azide) that induce cell death but not specifically by raising ROS levels (FIG. 6). mclk1 −/− cells were neither resistant nor hyper-sensitive to sodium azide and staurosporine but these cells were resistant to etoposide, anisomycin, all-trans retinoic acid, and serum withdrawal. However, upon treatment with sodium pyruvate, which partially rescues growth rate (TABLE 1), the resistance of the mclk1 −/− cells became indistinguishable from that of the mclk1 +/+ cells (FIG. 6). Thus, except for the resistance to menadione, the resistance to cell death-inducing agents appears to be a secondary effect of other mclk1 −/− phenotypes, presumably the slow growth rate.

TABLE 1
Phenotype of mclk1 −/− ES cells.

| | mclk1 +/+ | | | | mclk1 −/− | | | |
|---|---|---|---|---|---|---|---|---|
| | | | | Sodium | | | | Sodium |
| ES cell | Without | Sodium | | pyruvate | Without | Sodium | | pyruvate |
| phenotype^{a} | treatment | pyruvate | UQ9 | and UQ9 | treatment | pyruvate | UQ9 | and UQ9 |
| Resistance to | 22.7 ± 5.4 | 23 ± 7.5 | 24 ± 2.9 | 23.9 ± 6.9 | 68.4 ± 5.4 | 68.9 ± 6.5 | 91.3 ± 3.4 | 92.7 ± 4.2 |
| menadione | (100) | (101) | (105) | (105) | (304) | (304) | (403) | (403) |
| [% viability]^{b} | | | | | | | | |
| Oxygen consumption | 9.3 ± 0.6 | 9.1 ± 0.4 | 8.96 ± 0.8 | 8.96 ± 0.6 | 4.9 ± 0.5 | 7.9 ± 0.4 | 6.8 ± 0.7 | 8.8 ± 0.7 |
| [103 μl | (100) | (98) | (96) | (97) | (53) | (85) | (72) | (95) |
| O2/sec/μg of | | | | | | | | |
| protein] | | | | | | | | |
| Cell | 25.2 ± 5.2 | 25.0 ± 4.8 | 25.3 ± 5.5 | 25.7 ± 5.5 | 9.4 ± 1.5 | 18.9 ± 2.8 | 17.5 ± 0.4 | 26.8 ± 5.7 |
| multiplication | (100) | (99) | (100) | (102) | (39) | (75) | (69) | (106) |
| [×10⁵ cells]^{c} | | | | | | | | |
| LIF requirement | 43.3 ± 10.6 | 39.3 ± 12.7 | 39.0 ± 18.5 | 44.7 ± 11.0 | 293.3 ± 11.5 | 181.3 ± 3.2 | 193.7 ± 40.8 | 133.3 ± 10.1 |
| [% undifferentiated | (100) | (90) | (90) | (102) | (676) | (418) | (446) | (306) |
| colonies, | | | | | | | | |
| n = 300]^{d} | | | | | | | | |
| Basal ROS levels | 4.0 ± 0.5 | 4.1 ± 0.3 | 3.8 ± 0.2 | 3.6 ± 0.2 | 2.4 ± 0.3 | 2.3 ± 0.2 | 2.4 ± 0.3 | 2.3 ± 0.3 |
| [Fluorescence | (100) | (100) | (94) | (90) | (59) | (57) | (59) | (57) |
| Unit × 100/μg | | | | | | | | |
| protein] | | | | | | | | |
| Induced ROS levels | 11.5 ± 0.8 | 11.8 ± 1.2 | 11.5 ± 0.5 | 11.7 ± 1.1 | 8.0 ± 0.7 | 8.1 ± 1.1 | 6.7 ± 1.2 | 6.7 ± 1.1 |
| [Fluorescence | (100) | (102) | (100) | (101) | (69) | (70) | (57) | (57) |
| Unit × 100/μg | | | | | | | | |
| Protein] | | | | | | | | |
| DNA damage^{f} | 45.3 ± 8.4 | n.d. | n.d. | n.d. | 28.7 ± 3.2 | n.d. | n.d. | n.d. |
| [% cells with | (100) | | | | (63) | | | |
| tails] | | | | | | | | |
| Lipid peroxidation | 18.1 ± 0.6 | n.d. | n.d. | n.d. | 10.7 ± 1.5 | n.d. | n.d. | n.d. |
| MDA equivalents^{g} | (100) | | | | (59) | | | |
| (pmol/μg | | | | | | | | |
| protein) | | | | | | | | |
| Gluthatione levels | 8.8 ± 0.6 | n.d. | n.d. | n.d. | 11.8 ± 0.95 | n.d. | n.d. | n.d. |
| [pmol/μg] | (100) | | | | (134) | | | |
| Mitochondrial | 15.1 ± 1.6 | n.d. | n.d. | n.d. | 4.7 ± 0.2 | n.d. | 9.1 ± 0.5 | n.d. |
| complex II | (100) | | | | (31) | | (60) | |
| activity without | | | | | | | | |
| exogenous Q1 | | | | | | | | |
| [nmol/min/ | | | | | | | | |
| mg protein]^{h} | | | | | | | | |
| Mitochondrial | 61 ± 6.0 | n.d. | n.d. | n.d. | 23.1 ± 3.5 | n.d. | 39.0 ± 3.5 | n.d. |
| complex II | (100) | | | | (38) | | (64) | |
| activity with | | | | | | | | |
| exogenous Q1 | | | | | | | | |
| [nmol/min/ | | | | | | | | |
| mg protein]^{h} | | | | | | | | |

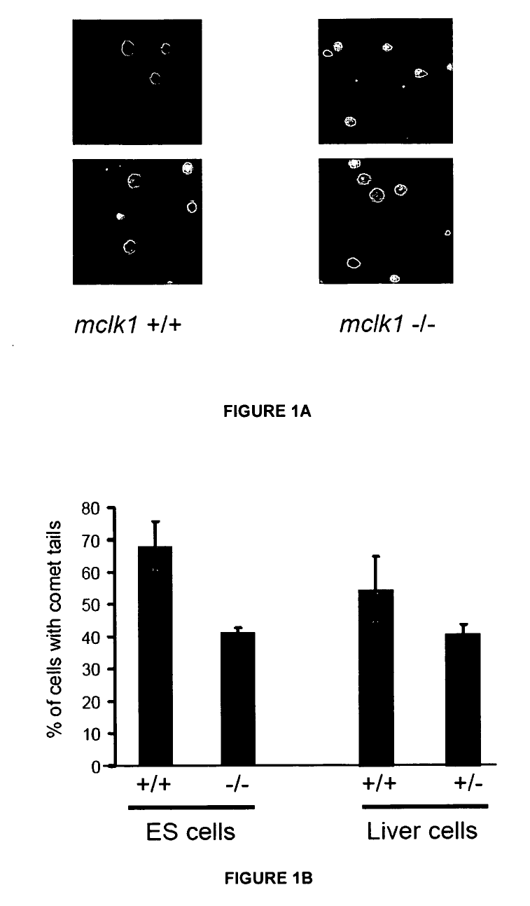

^{a}The number in brackets in each cell represents the phenotype as a percent of the wild-type phenotype (mclk1 +/+ ES cells).
^{b}Cell viability is expressed as the proportion of cells after 24 hours of growth in menadione (6 μM), with the number on day 1 being 1 × 10⁵.
^{c}Cell multiplication is expressed as the number of cells on day 5 of growth, with the number on day 1 being 1 × 10⁵.
^{d}LIF requirement is expressed as the percentage of colonies on day 3 of growth consisting of undifferentiated cells only when the cells are grown in medium with 0.008 M LIF.
^{e}Oxidative stress was induced by treatment with 0.1 mM FeSO4 and 0.2 mM sodium ascorbate.
^{f}DNA damage was measured by a ‘comet’ assay (see also FIG. 1). Cells were counted as damaged when a ‘comet tail’ could be seen, but the size of the tail was not taken into account; the experiment was repeated three times and the means and standard errors are given.
^{g}The measure of lipid peroxidation is by the TBARS assay and is given in malondialdehyde (MDA) equivalents.
^{h}Exogenous Q1 is added in vitro to increase the activities measured. Significantly, complex II activity is higher when the mclk1 −/− cells have grown in Q9-containing medium, even in the presence of an excess of Q1.

### Example 2

### Reduced Damage in MCLK1 −/− ES Cells

ROS are toxic molecules that damage proteins, lipids and nucleic acids. The inventors wondered therefore whether the mclk1 phenotype resulted in a decrease of oxidative damage. Oxidative damage to lipids was examined by the thiobarbituric acid-reactive substances (TBARS) assay (Janero, Free Radic. Biol. Med. 9: 515-40, 1990), and found to be significantly lower in mclk1 −/− cells than in control cells (TABLE 1). Damage to proteins was only examined indirectly by measuring the level of glutathione, a molecule that is crucial in protecting proteins from ROS damage (Dickinson and Forman, Biochem. Pharmacol. 64: 1019-26, 2002). Glutathione levels were elevated in the mclk1 −/− cells (TABLE 1). Glutathione levels can be reduced by high ROS, but glutathione synthesis can also be stimulated in response to ROS (Dickinson and Forman 2002). Thus, although the high levels of glutathione we observed likely is indicative of low protein damage in the mutant cells, it cannot be directly related to low ROS levels.

To examine DNA damage the comet assay (Collins, Mol. Biotechnol. 26: 249-61, 2004) was used, an in situ method that minimizes artefacts due to extraction procedures, and in which damaged DNA is visualized as a smear of DNA coming out of lysed nuclei under electrophoresis. DNA damage in mclk1 −/− cells was much less pronounced than that in the isogenic wild-type cells that were cultured in parallel (FIGS. 1a and 1b). Furthermore, the differences seen in FIG. 1b are likely to be an underestimate, as the numbers given do not take into account the sizes of the smears, which were systematically larger in the wild type cells (FIG. 1a).

### Example 3

### Long Lifespan of MCLK1 +/− Mice

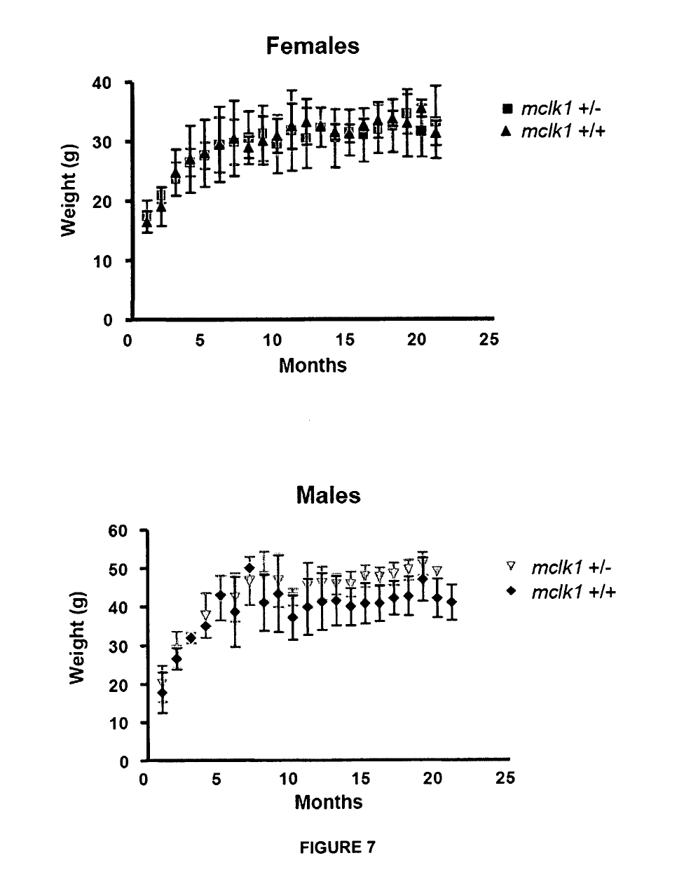

Given the inventors' observations of low levels of oxidative stress and DNA damage in mclk1 −/− cells, and the fact that reducing clk-1 activity prolongs the lifespan of nematodes (Wong et al., Genetics 139: 1247-59, 1995; Lakowski and Hekimi, Science 272: 1010-3, 1996), it was of interest to test the effect of reducing the activity mclk1 on the lifespan of mice. Although 2-day old mclk1 +/− heterozygous mice display a reduced level of the mCLK1 protein (Levavasseur et al., J. Biol. Chem. 276: 46160-4, 2001), they are fully viable. These mice are born at the expected frequency of ⅔ of the live progeny of heterozygous parents (168 (65%) mclk1 +/− and 89 (35%) mclk1 +/+ pups from 43 litters). The growth rates and the adult weights of +/− and +/+ females are similar, but adult male +/− might be somewhat heavier than +/+ (FIG. 7). The fertility of females is not different from that of control animals by various measures (Table 2).

TABLE 2
Fertility of mclk1 +/− females.

| | mclk1 +/+ | mclk1 +/− |
|---|---|---|
| Female fertile age (weeks)^{a} | 5.8 ± 0.5 (n = 5) | 5.7 ± 0.6 (n = 6) |
| Number of embryos^{a,b} | 8.5 ± 2.9 (n = 8) | 8.0 ± 3.1 (n = 4) |
| Number of live newborns^{c} | C57BI/6J: | C57BI/6J: |
| | 8.7 ± 2.0 (n = 13)^{d} | 9.3 ± 1.8 (n = 10) |
| | 129SV/j: | 129SV/j: |
| | 7.5 ± 2.7 (n = 8) | 5.8 ± 1.7 (n = 4) |
| | Balb/c: | Balb/c: |
| | 8.3 ± 2.7 (n = 12) | 7.3 ± 2.1 (n = 4) |
| Estrus cycle length at 6 | 5.0 ± 0.8 (n = 5) | 5.3 ± 0.9 (n = 4) |
| months (days)^{a} | | |

^{a}The genetic background of the mice was 129SV/j.
^{b}The number of embryos was determined by dissecting the embryos from the uteri of pregnant females and genotyping them at day E10.5.
^{c}The males used were mclk1 +/− for the mclk1 +/+ females, and mclk1 +/+ for the mclk1 +/− to obtain an equal genotype distribution for the pups.
^{d}The sample size is the number of litters of live newborns examined.

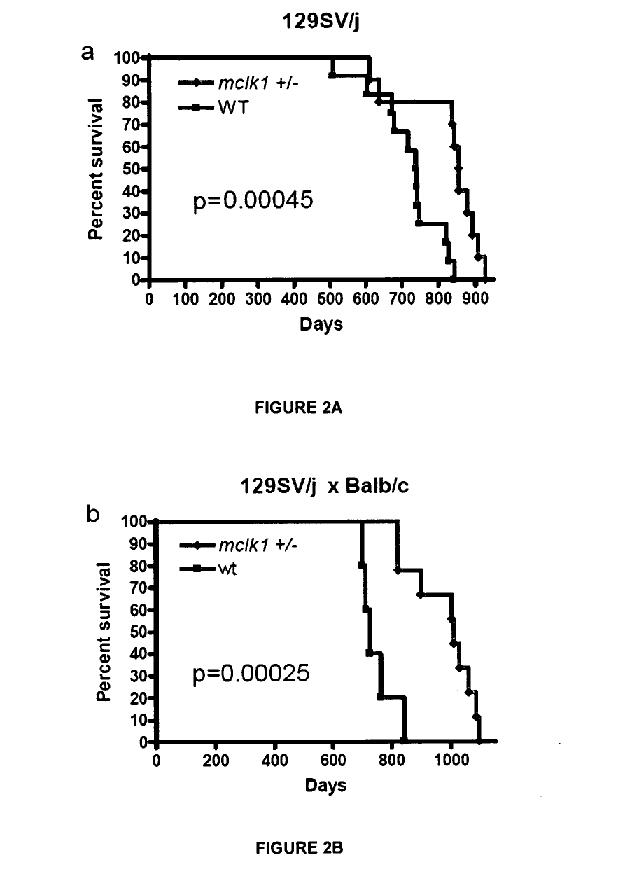

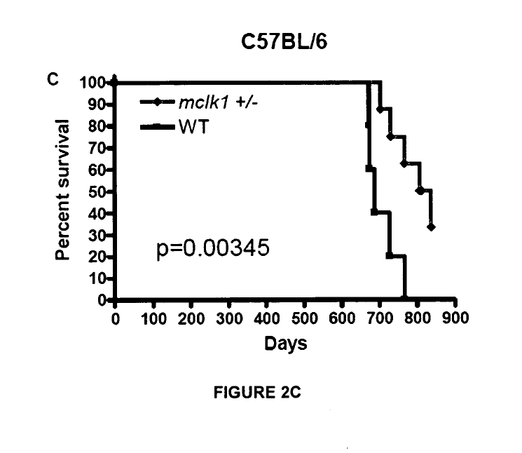

To date the inventors have examined the effect of mclk1 on lifespan in three genetic backgrounds: the study in the 129SV/j background and in F1 animals from a 129SV/j cross with Balb/c have been completed, and the study in the C57BL/6 background is ongoing (FIG. 2). The inventors analyzed survival by the Mantel-Haenszel test and found significantly greater survival of the mclk1 +/− in all three studies, p=0.00045, p=0.00025 and p=0.00345, respectively (FIG. 2). In the 129SV/j background the maximum lifespan was 928 days for the mclk1 +/− mice (n=10) and 843 days for the wild type animals (n=12), and mclk1 +/− mice lived on average 15% longer than their wild type littermates (824.8±103.8 vs 720.2±96.1 days). In the 129SV/j×Balb/c background, the maximum lifespan of mclk1 +/− animals (n=9) was 1092 days and only 843 days for their wild-type siblings (n=5). The mclk1+/− mice lived on average 31% longer than their wild type littermates (980.4±105.9 vs 749.8±57.2 days). Although the study in the C57BL/6 background is not complete, the available data shows a median survival of 686 days for mclk1 +/+ (n=5) and of 821.5 days for mclk1 +/− (n=8), a difference that is already significant at p=0.00345.

The 129SV/j animals tested were all females. The F1 and the C57BL/6 animals tested were both male and female. In the F1 study only three of the heterozygotes and one wild type animal were male. The average lifespans of the tested F1 females only (1019±98 days for mclk1 +/− versus 762±54 days for the wild type) were not meaningfully different from the average lifespans that include the males. There are both males and females in the C57BL/6 study and both sexes behave similarly. Currently, the median lifespan of the males is 726 days for mclk1 +/+ (n=3) and 837 days for mclk1 +/− (n=5) (p=0.026). Overall, it appears that the lifespan of males and females are similarly affected.

A number of facts indicate that the lifespan increases the inventors observe are robust: 1) the total number of mice in the three studies is significant (22 wild type and 27 heterozygotes), 2) all three studies show an increase lifespan for the mclk1 +/− animals, 3) the observed differences in each of the three studies are statistically significant, 4) the three experiments give similar results in spite of the differences of genetic background, 5) the three experiments were carried out independently, over different time periods, 6) the control wild-type animals tested were the siblings of the heterozygotes, 7) although the female data is more extensive, males and females show similar effects.

### Example 4

### Low DNA Damage in MCLK1 +/− Mice

The inventors tested the possibility that mclk1 +/− mice were experiencing lower levels of ROS damage to DNA by using the comet assay to compare the livers of mclk1 +/− to those of mclk1 +/+ animals (age range 14-18 months; n=7 for each genotype). The mclk1 +/− mice experience significantly (p<0.05) lower levels of DNA damage by this measure (FIG. 1b).

### Example 5

### Loss of MCLK1 Expression in the Liver of Aged Mice

The magnitude of the effect on lifespan of the mclk1 +/− heterozygous condition was surprising because, in a previous study, the inventors did not observe a reduced level of ubiquinone in young heterozygotes (Levavasseur et al., J. Biol. Chem. 276: 46160-4, 2001). If the phenotypic effects of reduced clk-1/mclk1 activity observed in worms and in ES cells are entirely mediated by a reduction of the level of ubiquinone, then one should not expect to observe an effect on the lifespan of the heterozygous mice. The inventors wondered if increased lifespan could be due to a phenomenon of loss-of-heterozygosity, and if old heterozygous mice contained populations of mclk1 −/− cells. The inventors' findings with mclk1 −/− ES cells suggested that homozygous somatic cells produced by spontaneous loss-of-heterozygosity might experience reduced oxidative stress, which could confer a growth or survival advantage resulting in expanded mclk1 −/− clones. The inventors chose to examine the liver for such a phenomenon because of the large regenerative potential of hepatocytes and other hepatic cell types, which can produce large clones in regenerating livers.

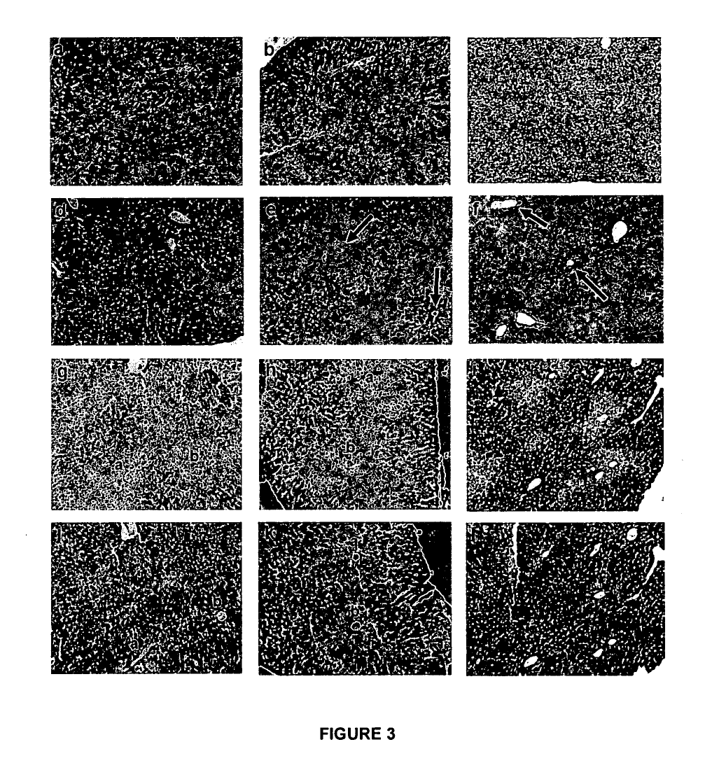

The inventors discovered the presence of large groups of cells that do not express mclk1 in the livers of every old mclk1 +/− animal examined (age range: 25-36 months; n=6), but not in the livers of either old mclk1 +/+ (age range: 25-27 months; n=3), or young mclk1 +/− or mclk1 +/+ animals (age range: 4-6 months: n=3 for each genotype) (FIG. 3). Cells lacked mclk1 at the protein level, as detected with a mCLK1-specific antiserum (Jiang et al., J. Biol. Chem. 276: 29218-25, 2001) (FIG. 3e), as well as at the RNA level, as demonstrated by in situ hybridization (FIG. 3f). In these cells, the inventors also observed a slight decrease in the expression of three mitochondrial markers: SOD2, cytochrome C and the subunit 1 of mitochondrial complex IV (FIG. 3j,k,l). This is consistent with the reduction in mitochondrial oxygen consumption, and the activity of mitochondrial complexes, that is observed in mclk1 −/− ES cells not supplemented with ubiquinone (TABLE 1).

### Example 6

### Loss-of-Heterozygosity at the MCLK1 Locus

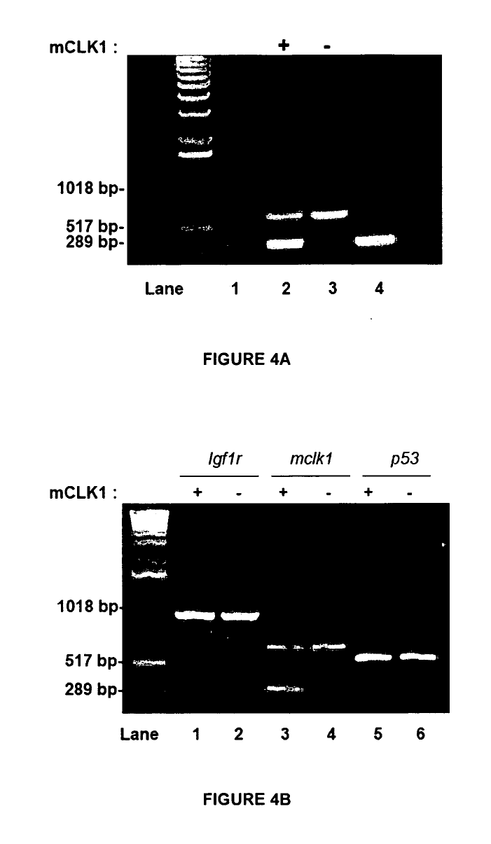

A classic mechanism to account for the total loss of expression of a gene in a subset of cells of a heterozygous (+/−) animal is loss-of-heterozygosity (LOH), the spontaneous mutational deletion of the wild-type allele (Thiagalingam et al., Curr. Opin. Oncol. 14: 65-72, 2002). To investigate whether this is the mechanism behind the appearance of mclk1 −/− clones, the inventors used laser-capture microdissection (LCM)(De Preter et al., Cancer Lett. 197: 53-61, 2003). Cells from liver sections in which mclk1 expressing and non-expressing areas were identified by immunocytochemistry, were obtained by LCM, and DNA extracted from the captured cells was submitted to whole-genome amplification by the multiple strand displacement amplification technique (Dean et al., Proc. Natl. Acad. Sci. USA 99: 5261-6, 2002). The amplified DNA was then analyzed by PCR for the presence of mclk1-, igf1r- and p53-specific sequences. mclk1 and igf1r are both on chromosome 7 and p53 is on chromosome 11. Cells from 8 mCLK1-expressing, and 8 mCLK1-negative clones were examined in this way. In 6/8 cases the inventors found that mCLK1-negative cells contained only sequences from the allele disrupted by targeted recombination (FIG. 4). In 2/8 cases, no mclk1-specific amplification from the mCLK1-negative cells was observed. However, in all cases the inventors could amplify mclk1-specific products from mCLK1-expressing clones. Furthermore, the inventors could always amplify igf1r- and p53-specific sequences from mCLK1-negative and mCLK1-expressing captured cells (FIG. 4b). In conclusion, the loss of expression of mCLK1 appears to be linked to the specific loss of the wild-type allele of mclk1.

### Example 7

### Clonal Expansion of MCLK1 −/− Cells

Strikingly, the distribution of mclk1 −/− cells was not random with respect to the main microanatomical compartment of the liver, the lobule, which is the region drained by a single vein of the microvasculature. In fact, the clones generally appeared to be of a similar size and frequently appeared to correspond to entire lobules (FIG. 3). Affected lobules were quite numerous, representing as much as 50% of the tissue in certain regions of the liver. The existence of large clones that have lost mclk1 expression suggests a mechanism in which loss of mclk1 expression confers a growth advantage to single hepatocytes, which over time allows their progeny to replace all hepatocytes in the lobule in which they originated.

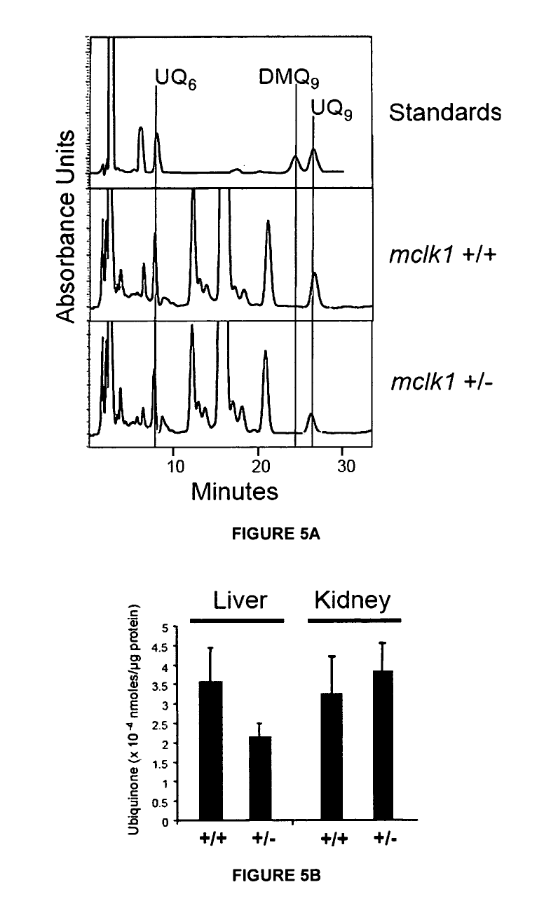

The inventors also found that the livers, but not the kidneys of relatively old mclk1 +/− animals (age range 14 to 22 months) contained less ubiquinone relative to protein than those of mclk1 +/+ animals (FIG. 5b). As no difference in ubiquinone content is observed in the liver of young animals (Levavasseur et al., J. Biol. Chem. 276: 46160-4, 2001), these findings are consistent with an age-dependent increase of liver cells that have lost mclk1 expression. Interestingly, the inventors did not observe the presence of DMQ in these livers (FIG. 5a). Nor did the inventors detect DMQ in any other organ. This shows that, in contrast to what is observed in ES cells, the UQ synthesis pathway is turned off in adult hepatocytes in the absence of the mCLK1 protein.

Discussion

Aging and Oxidative Stress

The inventors find that reducing the activity of mclk1 reduces ROS levels, oxidative stress, and oxidative damage in mouse cells, and prolongs the lifespan of whole animals. Such a correlation between lifespan and the level of oxidative stress and its consequences has frequently been observed and has led to the oxidative stress theory of aging. Decreased oxidative stress must at least be considered a marker for a physiological condition that favors increased lifespan. An increased resistance to some type of oxidative stress has been frequently found in association with increased lifespan in genetic models, including in the long-lived dwarf mice (Hauck et al., Horm. Metab. Res. 34: 481-486, 2002), igf1r +/− mice (Holzenberger et al., Nature 421: 182-7, 2003; Baba et al., J Biol Chem 280: 16417-26, 2005) and p66shc −/− mice (Migliaccio et al., Nature 402: 309-13, 1999; Nemoto and Finkel, Science 295: 2450-2, 2002; Napoli et al., Proc. Natl. Acad. Sci. USA 100: 2112-6, 2003). These results with mclk1 strengthen the generality of the observation that resistance to oxidative stress accompanies increased lifespan. In addition, the fact that reduction of mclk1 activity has similar ROS-reducing and lifespan-lengthening effects in mice and in nematodes, and that the mclk1 gene product is an enzyme involved in the production of a major cellular redox cofactor also suggests a causal link between ROS reduction and increased longevity.

Increased Fitness of mclk1 −/− Cells

The inventors find that in the livers of every old mclk1 +/− animal examined entire hepatic lobules have lost mclk1 expression. Hepatic lobules appear to be either entirely positive or entirely negative for mclk1 expression. This shows a model in which random mclk1 inactivation in a single cell of a lobule leads to clonal expansion within the microanatomical compartment of the lobule. It is reasonable to expect that the hepatocytes in which mclk1 are inactivated will have a number of properties in common with the mclk1 −/− ES cells. Thus, the observed phenomenon of clonal expansion might be due to increased resistance of these cells to age-dependent oxidative stress and apoptosis. As the capacity of the liver to regenerate decreases with age (Fry et al., J. Cell Physiol. 118: 225-32, 1984), the mclk1 −/− cells might be the only cells in these old livers that have sustained sufficiently little damage and are sufficiently resistant to stress to be capable of extensive propagation.

To date the inventors do not know when the mclk1 −/− clones arise during the life of the animals, except that it appears that none are observed at 5 months of age. Thus, it is possible that, rather than being the result of the response to acute age-related stresses, the clones arise gradually as the result of the normal process of cell turnover. Given the importance of ROS in the regulation of apopotosis, it is possible that the expected low level of ROS in mclk1 negative hepatocytes makes spontaneously arising mclk1 −/− and their descendants resistant to the physiological apoptosis that is part of normal cell turnover in tissues.

Loss-of-Heterozygosity at the mclk1 Locus

The phenomenon of loss-of-heterozygosity (LOH) can be observed in animals heterozygous for a loss-of-function mutation in a tumor suppressor gene (Knudson, Proc. Natl. Acad. Sci. USA 90: 10914-21, 1993; Devilee et al., Trends Genet. 17: 569-73, 2001). The cells that spontaneously lose the second allele of the gene, for example through the loss of an entire chromosome or a large section of a chromosome, escape normal growth controls and clonally expand into a tumor. LOH is but one of the consequences of the accumulation of somatic mutations that is one of the proposed mechanisms of aging (Hasty et al., Science 299: 1355-9, 2003). However, these results show that, under special circumstances, these normally deleterious processes could act favorably by inactivating the remaining wild-type allele in animals heterozygous for a gene whose normal activity limits lifespan.

Basis of the Increased Cellular Fitness

It has been a question as to whether the increase in lifespan of clk-1 mutants in C. elegans was due to the presence of DMQ (Jonassen et al., J. Biol. Chem. 277: 45020-7, 2002; Shibata et al., Science 302: 1779-82, 2003). Here the inventors find that the livers but not the kidneys of relatively old mclk1 +/− mice have lower levels of ubiquinone than those of mclk1 +/+ animals (FIG. 5b). This is presumably due to the presence of mclk1 −/− cells as such difference in ubiquinone content between genotypes is not observed in younger animals (Levavasseur et al., J. Biol. Chem. 276: 46160-4 2001). However, there is no detectable DMQ in these livers (FIG. 5a) or in any other organ of these animals. This shows that in old liver cells, in contrast to what was observed in ES cells, the entire pathway of ubiquinone biosynthesis is turned off when mCLK1 is absent, a phenomenon that is also observed in yeast (Marbois and Clarke, J. Biol. Chem. 271: 2995-3004, 1996). Thus, the low level of DNA damage in old mclk1 +/− livers, the apparent growth advantage of mclk1 −/− cells, and the increased lifespan of these animals cannot be due to the presence of DMQ.

The above considerations suggest that, if the growth advantage and the decreased DNA damage are due to low oxidative stress, then the ubiquinone normally present in wild-type cells is in fact contributing to oxidative stress, as has been suggested for nematodes (Larsen and Clarke, Science 295: 120-3, 2002). How could we explain the presence of deleterious amounts of ubiquinone in animal cells? The explanation likely hinges on the fact that ubiquinone has both prooxidant and antioxidant properties. For example, although ubiquinone is a membrane antioxidant, its function in the ubiquinone cycle of the respiratory chain promotes the formation of superoxide when it is in the ubisemiquinone state. Thus, the normal level of ubiquinone is likely a compromise between the need for antioxidant protection from acute stresses and its prooxidant role as co-factor. The amount of ubiquinone that is adequate to protect from acute oxidative stresses, such as can be brought about by heat stress, irradiation or transient anoxia, might in fact participate in creating chronic oxidative stress.

Molecular Basis of Increased Lifespan of mclk1 +/− Mice

The observations made above suggest two distinct possibilities for the increased lifespan of mclk1 +/− animals. The first possibility is that the presence of clones of mclk1 −/− cells could be sufficiently beneficial for the animal as a whole. This could be the case if there was net loss of cells in some organs without the presence of mclk1 +/− cells, or if these cells were somehow physiologically superior. However, all the animals examined were part of the inventors' aging study, and were examined shortly before natural death, with most organs in a state of relative deterioration. Therefore, although the inventors found clones only in the liver, the data for other organs such as the kidney and the gut was not of sufficient quality to be able to conclude firmly whether there were clones or not. Yet, even if mclk1 −/− clones can develop only in the liver, due to the regeneration potential of hepatocytes, this might be sufficient to result in increased lifespan thanks to the important role of the liver in digestion, detoxification and the regulation of circulating glucose levels.

The second possibility is that the presence of reduced amounts of mCLK1 protein in all the cells of the mclk1 +/− animals is the lifespan-lengthening factor. Studies in ES cells, in embryos, and in young animals (Levavasseur et al., J. Biol. Chem. 276: 46160-4, 2001), as well as the results presented here with the kidneys of old mclk1 +/− animals, suggest that mclk1 is recessive for ubiquinone biosynthesis. Yet it is possible that reduction of mclk1 expression might bring about an undetected minor reduction of ubiquinone levels, or a reduction in particular cell types, or during particular physiological conditions, that could be favorable for longevity by increasing resistance to damage at significant times and/or places.

Significance of Evolutionary Conservation

It is a broadly, if not universally, accepted view that aging is a consequence of the gradual accumulation of unrepaired molecular damage produced by endogenous processes or the environment. Thus, given that each species has its own physiology and environment, it is likely that there are species-specific processes that promote or protect from damage or damage accumulation. However, the evolutionary conservation of the longevity promoting effect of clk-1/mclk1 mutations indicates that there are also processes that are shared between animals of such disparate morphologies, physiologies and ecologies as worms and mice. This might have its basis in the universal conservation of the function of small molecular weight effectors such as ubiquinone and ROS.

What is claimed is:

1. An isolated cell from a clk-1 +/− animal, said cell having a clk-1 −/− genotype.

2. The cell of claim 1, characterized in that said cell consists of an embryonic stem cell.

3. The cell of claim 1, characterized in that said cell consists of a non-embryonic cell.

4. The cell of claim 3, characterized in that said non-embryonic cell is selected from the group consisting of a liver cell, a skin cell and intestine cell.

5. The cell of claim 3 or 4, characterized in that said non-embryonic cell is a stem cell.

6. The cell of claim 1, having at least one of the following properties:
low levels of reactive oxygen species,
increased resistance to oxidative stress dependent apoptosis, and
reduced oxidative damage.

7. The cell of claim 1, characterized in that said clk-1 +/− animal is in the latter half of its natural life span.

8. A composition for use in the treatment of an oxidative stress disorder, comprising an isolated cell as defined in any one of claims 1 to 7 and a pharmaceutically acceptable carrier.

9. A method for treating a subject or diseased tissue in need of treatment for an oxidative stress disorder, said method comprising administering a therapeutically effective amount of an isolated clk-1 −/− cell as defined in claim 1 or a composition as defined in claim 8.

10. The method of claim 9, characterized in that said isolated cell is isolated from a cell sample obtained from a donor.

11. The method of claim 9, characterized in that prior to administrating said clk-1 −/− cell, said cell is cultured in vitro.

12. The method of claim 9, wherein the isolated cell is derived from the same type of organ or tissue in need of treatment.

13. The method of claim 9, characterized in that said oxidative stress disorder is selected from the group consisting of amyotrophic lateral sclerosis (ALS), Alzheimer's Disease and age-related disorder.

14. A clk-1 +/− non-human animal comprising clk-1 −/− cells.
